(19) United States
(12) Patent Application Publication
(10) Pub. No.: US 2026/0347284 A1
(43) Pub. Date: Nov. 5, 2026

(54) RECYCLED CONTENT PARAXYLENE FROM RECYCLED CONTENT PYROLYSIS OIL

(71) Applicant: Eastman Chemical Company, Kingsport, TN (US)

(72) Inventors: Xianchun Wu, Longview, TX (US); Daryl Bitting, Longview, TX (US); Nick Allen Collins, Fall Branch, TN (US); Michael Gary Polasek, Richmond, TX (US); David Eugene Slivensky, Tatum, TX (US)

(73) Assignee: Eastman Chemical Company, Kingsport, TN (US)

(21) Appl. No.: 19/099,957

(22) PCT Filed: Jul. 20, 2023
(86) PCT No.: PCT/US2023/070539

§ 371 Date: Jan. 30, 2025

Related U.S. Application Data
(60) Provisional application No. 63/370,239, filed on Aug. 3, 2022.

(51) Int. Cl. C07C 5/27; C07C 6/12; C07C 51/16; C10G 57/00; C10G 67/02

(52) U.S. Cl. CPC C07C 5/2729; C07C 6/126; C07C 51/16; C10G 57/00; C10G 67/02; C10G 2300/1003; C10G 2300/1033; C10G 2400/30

(57) ABSTRACT

Processes and facilities for producing a recycled content hydrocarbon product directly or indirectly from waste plastic. Processing schemes are described herein for converting waste plastic (or hydrocarbon having recycled content derived from waste plastic) into useful intermediate chemicals and final products. In some aspects, recycled content aromatics (r-aromatics) can be processed to provide recycled content paraxylene (r-paraxylene), which can then be used to provide recycled content terephthalic acid (r-TPA) and/or recycled content polyethylene terephthalate (r-PET).

### BACKGROUND

Aromatic compounds such as benzene, toluene, and xylenes are important industrial chemicals used in a variety of applications. Para-xylene is used to form dicarboxylic acids and esters, which are key chemical feedstocks in the production of polyesters and aromatics-based plasticizers. Most conventional production routes for these materials utilize fossil fuel-derived feeds. Thus, it would be desirable to find additional synthesis routes for para-xylene and other aromatics that are sustainable, while also providing high-purity end products. Advantageously, the manufacture of such components can be carried out with existing equipment and facilities.

### SUMMARY

In one aspect, the present technology concerns a method of producing recycled-content p-xylene (r-p-xylene). A recycled-content pyoil stream (r-pyoil) and a crude oil stream are distilled in at least one distillation column to produce at least one recycled-content heavy hydrocarbons (r-heavy hydrocarbons)stream having a T₅₀ of greater than 400 °F. The average molecular weight of the r-heavy hydrocarbons stream is reduced to produce a recycled-content naphtha (r-naphtha) stream and/or a recycled-content pyrolysis gasoline (r-pyrolysis gasoline) stream. At least a portion of the r-naphtha stream and/or at least a portion of the r-pyrolysis gasoline stream directly or indirectly are reformed in a reformer to produce a recycled-content reformate (r-reformate) comprising r-p-xylene.

In one aspect, the present technology concerns a method of producing r-p-xylene. An r-pyoil stream and a crude oil stream are distilled within an atmospheric distillation column to produce an r-atmospheric distillation column bottoms stream. At least a portion of the r-atmospheric distillation column bottoms stream is distilled within a vacuum distillation column to produce an r-heavy vacuum gas oil stream. The average molecular weight of at least a portion of the r-heavy vacuum gas oil stream is reduced within a cracker to produce an r-naphtha stream and/or an r-pyrolysis gasoline stream. At least a portion of the r-naphtha and/or pyrolysis gasoline streams directly or indirectly are reformed in a reformer to produce an r-reformate comprising r-p-xylene.

In one aspect, the present technology concerns a method of producing r-p-xylene. A recycled-content pyoil stream and a crude oil stream are distilled within an atmospheric distillation column and/or a vacuum distillation column to produce an r-atmospheric gas oil stream and/or a r-light vacuum gas oil stream. At least a portion of at least one of the r-light vacuum gas oil stream and the r-atmospheric gas oil stream are optionally hydrotreated to produce a hydrotreated stream. The average molecular weight of at least a portion of the hydrotreated stream or at least a portion of at least one of the r-light vacuum gas oil stream and the r-atmospheric gas oil stream are reduced within a fluidized catalytic cracker to produce an r-naphtha stream. Optionally, at least a portion of the r-naphtha stream is hydrotreated to produce a hydrotreated r-naphtha stream. At least a portion of the r-naphtha stream or the hydrotreated r-naphtha stream is reformed to produce an r-reformate comprising r-p-xylene.

In one aspect, the present technology concerns a method of producing r-p-xylene. An r-pyoil stream and a crude oil stream are distilled within an atmospheric distillation column and a vacuum distillation column to produce an r-atmospheric gas oil stream, an r-light vacuum gas oil stream, and an r-heavy vacuum gas oil stream. The average molecular weight of the r-heavy vacuum gas oil stream is reduced within a hydrocracker to produce a first r-naphtha stream. At least a portion of at least one of the r-light vacuum gas oil stream and the r-atmospheric gas oil stream are hydrotreated to produce a hydrotreated stream, and the average molecular weight of the hydrotreated stream is reduced within a fluidized catalytic cracker to produce a second r-naphtha stream. Optionally, at least a portion of the first and/or second r-naphtha streams are hydrotreated to produce a hydrotreated r-naphtha stream. At least a portion of the first and/or second r-naphtha streams and/or the optional hydrotreated r-naphtha stream is reformed to produce an r-reformate comprising r-p-xylene.

In one aspect, the present technology concerns a method of producing r-p-xylene comprising directly or indirectly feeding an r-naphtha stream and/or at least a portion of an r-pyrolysis gasoline stream to a reformer to produce an r-reformate comprising r-p-xylene. At least a portion of the r-naphtha stream and/or r-pyrolysis gasoline stream is obtained by distilling an r-pyoil stream and a crude oil stream in at least one distillation column to produce at least one r-heavy hydrocarbons stream having a T₅₀ of greater than 400 °F. The average molecular weight of the r-heavy hydrocarbons stream is reduced to produce the r-naphtha stream and/or r-pyrolysis gasoline stream.

In one aspect, the present technology concerns a method of producing r-p-xylene comprising feeding an r-reformate and/or an r-pyrolysis gasoline stream to an aromatics complex. At least a portion of the r-reformate and/or r-pyrolysis gasoline stream are obtained by distilling an r-pyoil stream and a crude oil stream in at least one distillation column to produce at least one r-heavy hydrocarbons stream having a T₅₀ of greater than 400 °F. The average molecular weight of the r-heavy hydrocarbons stream is reduced to produce an r-naphtha stream and/or the r-pyrolysis gasoline stream. At least a portion of the r-naphtha stream and/or r-pyrolysis gasoline stream directly or indirectly are is reformed in a reformer to produce the r-reformate comprising r-p-xylene.

In one aspect, the present technology concerns a method of producing an r-terephthalic acid comprising oxidizing an r-p-xylene. The r-p-xylene is obtained by distilling an r-pyoil stream and a crude oil stream in at least one distillation column to produce at least one r-heavy hydrocarbons stream having a T₅₀ of greater than 400 °F. An average molecular weight of the r-heavy hydrocarbons stream is reduced to produce an r-naphtha stream and/or an r-pyrolysis gasoline stream. At least a portion of the r-naphtha stream and/or r-pyrolysis gasoline stream directly or indirectly are reformed in a reformer to produce an r-reformate comprising the r-p-xylene.

In one aspect, the present technology concerns a method of producing r-p-xylene. An r-pyoil stream and a crude oil stream are distilled in at least one distillation column to produce an r-heavy naphtha stream. Optionally, the r-heavy naphtha stream is hydrotreated to produce a hydrotreated r-heavy naphtha stream. At least a portion of the r-heavy naphtha stream or hydrotreated r-heavy naphtha stream is reformed in a reformer to produce an r-reformate comprising r-p-xylene.

### BRIEF DESCRIPTION OF THE DRAWINGS

FIG. 1 is a schematic process flow diagram illustrating the main processes/facilities in a system for providing recycled content hydrocarbon products, including r-paraxylene according to various embodiments of the present technology;

FIG. 2 is a schematic process flow diagram illustrating the main processes/facilities in a system for providing recycled content hydrocarbon products including r-paraxylene according to further embodiments of the present technology;

FIG. 3 is a schematic process flow diagram illustrating the main steps/zones in an aromatics complex suitable for use in the systems illustrated in FIGS. 1 and 2;

FIG. 4a is a block flow diagram illustrating the main steps of a process for making recycled content aromatics (r-aromatics) and recycled content paraxylene (r-paraxylene), and optionally, a recycled content chemical compound from the r-paraxylene, wherein the r-aromatics (and r-paraxylene and r-chemical compound) have physical content from one or more source materials; and

FIG. 4b is a block flow diagram illustrating the main steps of a process for making recycled content aromatics (r-aromatics) and recycled content paraxylene (r-paraxylene), and optionally, a recycled content chemical compound from the r-paraxylene, wherein the r-aromatics (and r-paraxylene and r-chemical compound) have credit-based recycled content from one or more source materials.

### DETAILED DESCRIPTION

We have discovered new methods and systems for producing paraxylene and organic chemical compounds formed by directly processing paraxylene or its derivatives, including, for example, organic chemical compounds such as terephthalic acid and polyethylene terephthalate. More specifically, we have discovered a process and system for producing paraxylene where recycled content from waste materials, such as waste plastic, are applied to paraxylene (or its derivatives) in a manner that promotes the recycling of waste plastic and provides paraxylene (or other organic chemical compounds) with substantial amounts of recycled content.

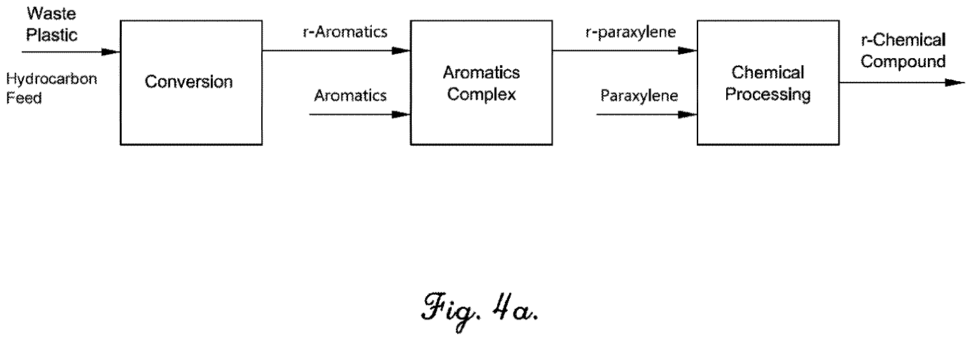

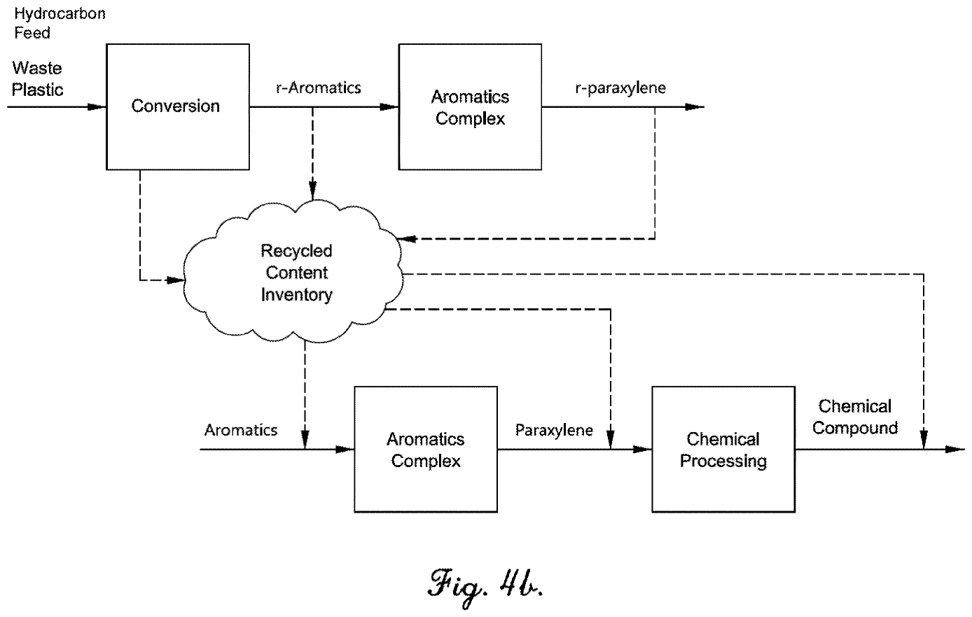

Turning initially to FIGS. 4a and 4b, paraxylene is formed by processing a predominantly aromatics stream in an aromatics complex to provide a stream including at least 85, at least 90, at least 92, at least 95, at least 97, or at least 99 weight percent paraxylene. The paraxylene stream can undergo one or more additional processing steps to provide at least one organic chemical compound derived from paraxylene. Examples of such organic chemical compounds include, but are not limited to, terephthalic acid, polymers such as polyethylene terephthalate, and other related organic chemical compounds.

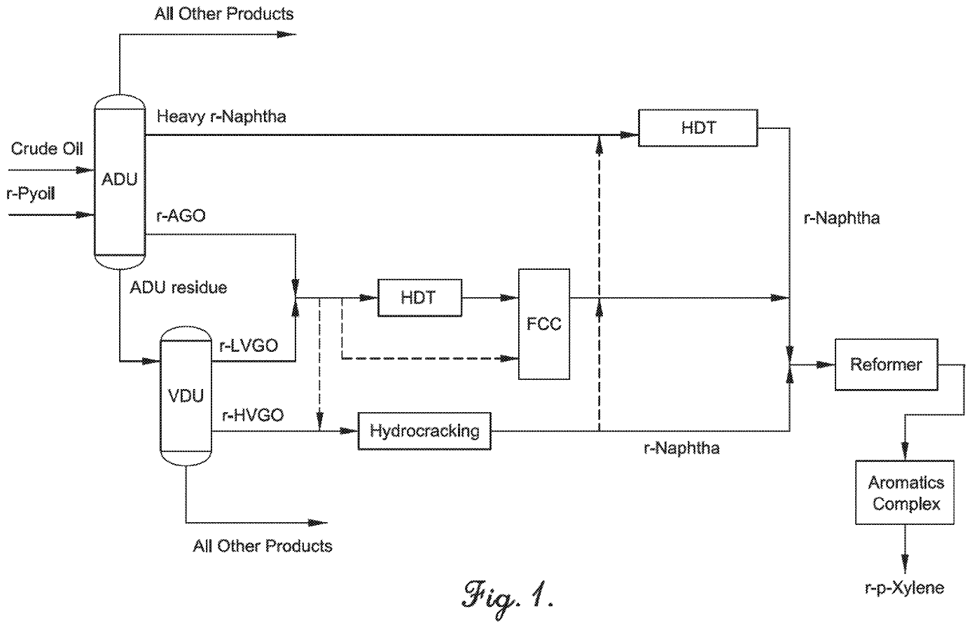

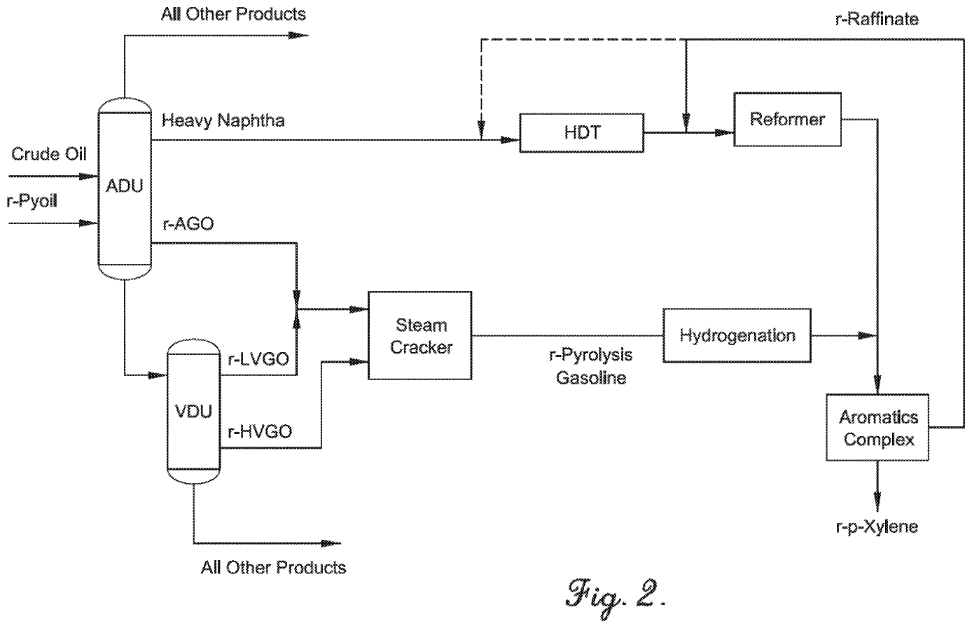

As generally shown in FIGS. 4a and 4b, a stream of waste plastic processed in one or more conversion facilities may provide the aromatics stream, which can be processed to form the paraxylene stream. The recycled content in the paraxylene stream can be physical and may directly originate from waste plastic or an intermediate hydrocarbon stream formed by processing waste plastic (not shown in FIG. 1 or 2), and/or the recycled content may be credit based and can be applied to a target stream in the aromatics complex and/or chemical processing facilities.

The aromatics (or paraxylene or organic chemical compound) streams can have a total recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. Similarly, the r-TPA and/or r-PET or even the r-aromatics stream can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. The recycled content in one or more of these streams can be physical recycled content, credit-based recycled content, or a combination of physical and credit-based recycled content.

Turning initially to FIG. 4a, in one embodiment or in combination with one or more embodiments mentioned herein, at least a portion of the recycled content in the aromatics and/or paraxylene stream (or in the organic chemical compound product stream) can be physical (direct) recycled content. This recycled content may originate from a waste plastic stream. The waste plastic stream is ultimately converted in one or more conversion facilities (e.g., a pyrolysis facility, a refinery, a steam cracking facility, and/or a molecular reforming facility and methanol-to-aromatics facility), which is processed (alone or with a non-recycled content aromatics stream) as described herein to provide an r-paraxylene stream. The r-paraxylene stream can then be further processed (along or in combination with a non-recycled content paraxylene stream) to provide a recycled content organic chemical compound, including, but not limited to, recycled content terephthalic acid (r-TPA), recycled content polyethylene terephthalate (r-PET), and one or more additional recycled content organic chemical compounds (r-organic chemical compounds).

The amount of physical recycled content in the target product (e.g. composition, r-aromatics or r-paraxylene or r-organic chemical compound) can be determined by tracing the amount of waste plastic material processed along a chain of chemical pathway(s) and ending with the moiety or portion of the target product attributable to the waste plastic chemical pathway. As used herein, a moiety can be a portion the atoms and their structure of a target product and can also include the entire chemical structure of the target product and does not necessarily require the inclusion of a functional group. For example, a moiety of p-xylene can include the aromatic ring, a portion of the aromatic ring, the methyl groups, or the entire p-xylene molecule. The chemical pathway includes all chemical reactions and other processing steps (e.g., separations) between the starting materials (e.g., waste plastic) and the moiety in the target product attributable to the chemical pathway originating in waste plastics. For example, the chemical pathway for the r-aromatics can include pyrolysis, optionally refining and/or stream cracking, and/or molecular reforming and methanol synthesis and conversion. The chemical pathway for the r-paraxylene can further include processing in the aromatics complex, and the chemical pathway for the r-organic chemical compound may include a variety of additional steps, such as, for example, oxidation, polymerization, etc., depending on the specific r-organic chemical compound. A conversion factor may be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of credit-based recycled content in a target product (e.g. compositions, r-aromatics or r-paraxylene or r-organic chemical compounds) can be determined by calculating the mass weight percent of a target moiety in a target product and attributing a recycle content credit to the target product in any amount up to the mass weight percent of the target moiety in the target product as a maximum. The credit based recycle content that is eligible to be applied to the target product is determined by tracing the waste plastic material along a chain of chemical pathway(s) and ending with the same moiety as target moiety in the target product. Thus, the credit based recycle content can be applied to a variety of different target products having the same moiety even though the products are made by entirely different chemical pathways provided that the credit applied is obtained from waste plastic and the waste plastic ultimately undergoes at least one chemical pathway originating from waste plastic and ending in the target moiety. For example, if a recycle content credit is obtained from waste plastic and booked into a recycle content inventory, and there exists chemical pathways at the facility capable of processing the waste plastic through to a target moiety such as p-xylene (e.g. a pyrolysis reactor effluent to a crude distillation column to a hydrotreater to a reformer to an aromatics complex that isolates p-xylene), the recycle content credit is then a type eligible to apply to any p-xylene molecule made by any chemical pathway, including the one existing at the facility and/or to the p-xylene portion of a pyrolysis gasoline stream composition obtained from a steam cracker and gasoline fractionator. As with physical recycled content, a conversion factor may or may not be associated with each step along the chemical pathway. Additional details on credit-based recycled content are provided below.

The amount of recycled content applied to the r-aromatics (or r-paraxylene or r-organic chemical compound) can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-aromatics (or r-paraxylene or r-organic chemical compound).

Turning now to FIG. 4b, one embodiment where the r-organic chemical compound (or r-paraxylene) includes credit-based recycled content, is provided. Recycled content credits from waste plastic are attributed to one or more streams within the facility. For example, the recycled content credits derived from waste plastics can be attributed to the aromatics stream fed to the aromatics complex, or to any of the products separated and isolated in the aromatics complex, such as to the paraxylene stream. Alternatively, or in addition, recycled content credits obtained from one or more intermediate streams within the conversion facility and/or aromatics complex can also be attributed to one or more products, such as paraxylene, within the facility, depending on the specific configuration of the system. Further, recycled content credits from one or more of these streams may also be attributed to the organic chemical compound stream, as shown in FIG. 4b.

As such, the waste plastic stream, or the r-aromatics stream and r-paraxylene streams (and any recycled content intermediate streams not shown in FIG. 4b) not made at the facility or purchased or acquired, can each act as a “source material” of recycled content credits. The aromatics fed to the aromatics complex, the paraxylene product or any other products separated and/or isolated from the aromatics complex, the paraxylene transferred (including sales) or fed to the chemical processing facility, any intermediate streams not shown, and even the organic chemical compound, can each act as a target product to which the recycled content credits are attributed. In one embodiment or in combination with any embodiment mentioned herein, the source material has physical recycled content and the target product has less than 100 percent physical recycled content. For example, the source material can have at least 10, at least 25, at least 50, at least 75, at least 90, at least 99, or 100 percent physical recycled content and/or the target product can have less than 100, less than 99, less than 90, less than 75, less than 50, less than 25, less than 10, less than 1 percent, or no physical recycled content.

The ability to attribute recycled content credits from a source material to a target product removes the co-location requirement between the facility making the source material (with physical recycled content) and the facility making the aromatics or products receiving recycle content value (e.g. paraxylene or organic chemical compound). This allows a chemical recycling facility/site in one location to process waste material into one or more recycled content source materials and then apply recycled content credits from those source materials to one or more target products being processed in existing commercial facilities located remotely from the chemical recycling facility/site, optionally within the same Family of Entities, or to associate a recycle content value with a product that is transferred to another facility, optionally owned by a different entity that can deposit the recycle content credit into its recycle content inventory one the product is receiving, purchased, or otherwise transferred. Further, the use of recycled content credits allows different entities to produce the source material and the aromatics (or paraxylene or organic chemical compound). This allows efficient use of existing commercial assets to produce the aromatics (or paraxylene or organic chemical compound). In one or more embodiments, the source material is made at a facility/site that is at least 0.1, at least 0.5, at least 1, at least 5, at least 10, at least 50, at least 100, at least 500, or at least 1000 miles from the facility/site where the target product is used to make the aromatics (or paraxylene or organic chemical compound).

The attributing of recycled content credits from the source material (e.g., the r-aromatics from the conversion facility) to the target product (e.g., an aromatics stream fed to an aromatics complex) can be accomplished by transferring recycled content credits directly from the source material to the target product. Alternatively, as shown in FIG. 4b, recycled content credits can be applied from any of the waste plastic, r-aromatics, and r-paraxylene (when present) to the aromatics, paraxylene, or organic chemical compound via a recycled content inventory.

When a recycled content inventory is used, recycled content credits from the source material having physical recycled content (e.g., the waste plastic, the r-aromatics, and optionally the r-paraxylene shown in FIG. 4b) are booked into the recycled content inventory. The recycled content inventory can also contain recycled content credits from other sources and from other time periods. In one embodiment, recycled content credits in the recycled content inventory correspond to a moiety, and the recycle content credit is applied or assigned to the same a target products containing a target moiety, and the target moiety is either (i) not chemically traceable through chemical pathways used to for generating the recycle content credit or (ii) is chemically traceable through chemical pathways used for generating the recycle content credit. Chemical traceability is achieved when atoms from a source material such as waste plastic can be theoretically traced to one or more atoms in the target moiety of a target product through each chemical pathway to obtain that atom(s) in the target moiety.

In some embodiments, there may be a periodic (e.g., annual or semi-annual) reconciliation between waste plastic credits deposited in the recycled content inventory and the mass of waste plastic processed. Such reconciliations may be performed by an appropriate entity at an interval consistent with rules of the certification system in which the producer is participating.

In one embodiment, once recycled content credits have been attributed to the target product (e.g., the aromatics stream, the paraxylene stream, or any intermediate stream not shown), the amount of the credit-based recycled content allocated to the organic chemical compound (e.g., TPA, PET, or other organic chemical compound) is calculated by the mass proportion of atoms in the target product that are chemically traceable to the source material. In another embodiment, a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway. However, if desired, the amount of recycle content applied to a target product can be more than the mass proportion of the target moiety chemically traceable to the waste plastic source material. The target product can receive up to 100% recycle content even though the mass proportion of atoms in the target moiety that is chemically traceable to a recycle source material, such as mixed plastic waste stream, is less than 100%. For example, if the target moiety in a product represents only 30 wt. % of all atoms in a target product that are chemically traceable to a mixed plastic waste stream, the target product can nevertheless receive more than 30% recycle content value, up to 100% if desired. While such application would violate chemical traceability for the full value of the amount of recycle content in a target product back to a waste plastic source, the particular amount of recycle content value applied to a target product will depend on the rules of a certification system that the producer participates in.

As with the physical recycled content, the amount of credit-based recycled content applied to the r-aromatics (or r-paraxylene or r-organic chemical compound) can be determined using one of variety of methods, such as mass balance, for quantifying, tracing, and allocating recycled content among various products in various processes. In certain embodiments the method of quantifying, tracing, and allocating recycled content is overseen by a certification entity that confirms the accuracy of the method and provides certification for the application of recycled content to the r-aromatics (or r-paraxylene or r-organic chemical compound).

The r-aromatics (or r-paraxylene or r-organic chemical compound) can have 25 to 90, 40 to 80, or 55 to 65 percent credit-based recycled content and less than 50, less than 25, less than 10, less than 5, or less than 1 percent physical recycled content. In certain embodiments, the r-aromatics (or r-paraxylene or r-organic chemical compound) can have at least 10, at least 25, at least 50, or at least 65 percent and/or not more than 90, not more than 80, or not more than 75 percent credit-based recycled content from one or more of the r-aromatics and/or r-paraxylene, individually.

In one or more embodiments, the recycled content of the r-aromatics (or r-paraxylene or r-organic chemical compound) can include both physical recycled content and credit-based recycled content. For example, the r-aromatics (or r-paraxylene or r-organic chemical compound) can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

We have discovered a method for producing a recycled content hydrocarbon product from hydrocarbon streams with recycled content derived from waste plastic. More specifically, hydrocarbon streams formed by the pyrolysis or cracking of waste plastic can be further processed in a petroleum refinery and/or cracking facility and/or reforming facility to provide recycled content aromatics, which are further processed in an aromatics complex to provide purified streams of recycled content benzene (r-benzene), recycled content toluene (r-toluene), and recycled content xylene (r-xylene), including recycled content paraxylene (r-pX). All or a portion of the r-pX can then be further processed to form additional recycled content chemicals, such as recycled content terephthalic acid (r-TPA) and/or recycled content polyethylene terephthalate (r-PET).

Turning initially to FIG. 1, a process and facility for use in forming a recycled content hydrocarbon product is provided. Note, that for convenience and simplicity, only the product streams described in the following description have been illustrated in the Figures. It is understood that a facility for use in forming a recycled content hydrocarbon product, including any separation units and reactor units contained therein, may produce additional product streams besides those illustrated. Specifically, the system illustrated in FIG. 1 can form recycled content paraxylene (r-pX) from one or more streams having recycled content from waste plastic. The system shown in FIG. 1 includes a refinery comprising one or more distillation units, one or more cracking facilities, such as a fluidized catalytic cracker and a hydrocracker, a reforming facility, and an aromatics complex. Optionally, at least a portion of the r-pX produced in the aromatics complex can be further processed and oxidized to form recycled content terephthalic acid (r-TPA) in a TPA production facility (not shown) and at least a portion of the r-TPA can be polymerized to form recycled content polyethylene terephthalate (r-PET) (not shown). The r-pX formed as described herein may be used in other applications not described herein.

In one embodiment or in combination with any embodiments mentioned herein, a stream having a recycled content used as a feed to the facility depicted in FIG. 1, e.g., pyrolysis oil, is produced from waste plastic within a chemical recycling facility. Chemical recycling facilities are not the same as mechanical recycling facilities. As used herein, the terms “mechanical recycling” and “physical recycling” refer to a recycling process that includes a step of melting waste plastic and forming the molten plastic into a new intermediate product (e.g., pellets or sheets) and/or a new end product (e.g., bottles). Generally, mechanical recycling does not substantially change the chemical structure of the plastic being recycled. The chemical recycling facilities described herein may be configured to receive and process waste streams from and/or that are not typically processable by a mechanical recycling facility.

In one embodiment or in combination with any embodiments mentioned herein, at least two, at least three, at least four, at least five, at least six, or all of the chemical recycling facility, the refinery, the one or more cracking facilities, the reforming facility, the aromatics complex, and the optional TPA production facility and the optional PET facility may be co-located. As used herein, the term “co-located” refers to the characteristic of at least two objects being situated on a common physical site, and/or within 5, within 3, within 1, within 0.75, within 0.5, or within 0.25 miles of each other, measured as a straight-line distance between two designated points. Alternatively, any product stream depicted in the Figures can be produced at one location and then transported by pipeline, truck, rail, or ship to another location for continued processing.

When two or more facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

Additionally, one or more, two or more, three or more, four or more, five or more or all, of the chemical recycling facility, the refinery, the one or more cracking facilities, the reforming facility, the aromatics complex, the TPA production facility, and the PET production facility may be commercial-scale facilities. For example, in one embodiment or in combination with any embodiments mentioned herein, one or more of these facilities/steps can accept one or more feed streams at a combined average annual feed rate of at least 500, at least 1000, at least 1500, at least 2000, at least 5000, at least 10,000, at least 50,000, or at least 100,000 pounds per hour, averaged over one year. Further, one or more of the facilities can produce at least one recycled content product streams at an average annual rate of at least 500, or at least 1000, at least 1500, at least 2000, at least 2500, at least 5000, at least 10,000, at least 50,000, or at least 75,000 pounds per hour, averaged over one year. When more than one r-product stream is produced, these rates can apply to the combined rate of all r-products.

One or more, two or more, three or more, four or more, five or more, or all, of the chemical recycling facility, the refinery, the one or more cracking facilities, the reforming facility, the aromatics complex, the TPA production facility, and the PET production facility can be operated in a continuous manner. For example, each of the steps or processes within each of the facilities and/or the process amongst the facilities may be operated continuously and may not include batch or semi-batch operation. In one embodiment or in combination with any embodiments mentioned herein, at least a portion of one or more of the facilities may be operated in a batch or semi-batch manner, but the operation amongst the facilities may be continuous overall.

In one embodiment or in combination with any embodiments mentioned herein, a mixed waste plastic can be introduced into a chemical recycling facility, which includes a pyrolysis facility. Within the pyrolysis facility, the mixed waste plastic may be pyrolyzed to form at least one recycled content pyrolysis effluent stream. The chemical recycling facility may also include a plastics processing facility (not shown) for separating a stream of mixed plastic waste into a predominantly polyolefin (PO) waste plastic and a predominantly non-PO waste plastic, which typically includes waste plastics such as polyethylene terephthalate (PET), polyvinyl chloride (PVC), and others. In addition, when present, the plastics processing facility can also remove other non-plastic components, such as glass, metals, dirt, sand, and cardboard from the incoming waste stream.

Within the pyrolysis facility, the waste plastic stream is pyrolyzed in at least one pyrolysis reactor. The pyrolysis reaction involves chemical and thermal decomposition of the waste plastic introduced into the reactor. Although all pyrolysis may be generally characterized by a reaction environment that is substantially free of oxygen, pyrolysis processes may be further defined by other parameters such as the pyrolysis reaction temperature within the reactor, the residence time in the pyrolysis reactor, the reactor type, the pressure within the pyrolysis reactor, and the presence or absence of pyrolysis catalysts.

The feed to the pyrolysis reactor can comprise, consists essentially of, or consists of waste plastic, and the feed stream can have a number average molecular weight (Mn) of at least 3000, at least 4000, at least 5000, or at least 6000 g/mole. If the feed to the pyrolysis reactor contains a mixture of components, the Mn of the pyrolysis feed is the average Mn of all feed components, based on the weight of the individual feed components. The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.). The feed to the pyrolysis reaction can also comprise less than 5, less than 2, less than 1, or less than 0.5, or about 0.0 weight percent of a co-feed stream, including steam and/or sulfur-containing co-feed streams. In other cases, steam fed to the pyrolysis reactor can be present in amounts of up to 50 weight percent.

The pyrolysis reaction can involve heating and converting the waste plastic feedstock in an atmosphere that is substantially free of molecular oxygen or in an atmosphere that contains less oxygen relative to ambient air. For example, the atmosphere within the pyrolysis reactor may comprise not more than 5, not more than 4, not more than 3, not more than 2, not more than 1, or not more than 0.5 weight percent of molecular oxygen.

The pyrolysis reaction in the reactor can be thermal pyrolysis, which is carried out in the absence of a catalyst, or catalytic pyrolysis, which is carried out in the presence of a catalyst. When a catalyst is used, the catalyst can be homogenous or heterogeneous and may include, for example, certain types of zeolites and other mesostructured catalysts.

The pyrolysis reactor may have any suitable design and can comprise a film reactor, a screw extruder, a tubular reactor, a stirred tank reactor, a riser reactor, a fixed bed reactor, a fluidized bed reactor, a rotary kiln, a vacuum reactor, a microwave reactor, or an autoclave. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds. The feed and/or lift can also include light hydrocarbons, such a methane, or hydrogen, and these gases may be used alone or in combination with steam.

A stream of recycled content pyrolysis effluent (r-pyrolysis effluent) removed from the reactor can be separated in a separation zone to provide a recycled content pyrolysis vapor (r-pyrolysis vapor) stream and a recycled content pyrolysis residue (r-pyrolysis residue) stream. The r-pyrolysis vapor can include a range of hydrocarbon materials and may comprise both recycled content pyrolysis gas (r-pygas) and recycled content pyrolysis oil (r-pyoil). In some embodiments, the pyrolysis facility may include an additional separation zone to separate the r-pyoil and r-pygas into separate streams. Alternatively, the entire stream of r-pyrolysis vapor may be withdrawn from the pyrolysis facility and routed to one or more downstream processing facilities.

Referring again to FIG. 1, at least a portion of the r-pyoil can be introduced into a refinery, along with a quantity of crude oil, wherein it can undergo one or more processing steps to provide at least one heavy hydrocarbons stream having a T₅₀ of greater than 400 °F, greater than 450 °F, greater than 500 °F, or greater than 520 °F, greater than 610 °F, greater than 800 °F, or greater than 950 °F, but less than 1050 °F, less than 950 °F, less than 800 °F, or less than 610 °F. In addition, a naphtha stream (r-naphtha), such as a heavy naphtha stream, having a T₅₀ of at least 90 °F, at least 120 °F, at least 150 °F, at least 180 °F, at least 190 °F, but less than 400 °F, less than 380 °F, less than 350 °F, less than 325 °F is also produced, as well as other recycled content hydrocarbon streams. Examples of suitable processing steps include, but are not limited to, distillation or other separation steps as well as chemical processing such as thermal and/or catalytic cracking or other reactions such as reforming and isomerization.

FIG. 1 is a schematic diagram illustrating the main steps or zones in a refining facility, or refinery, suitable for processing at least one hydrocarbon stream including recycled content derived from waste plastic. It should be understood that other processing steps may exist and/or other recycled content hydrocarbon streams may be produced in the refinery shown in FIG. 1. The steps, zones, and process streams illustrated in FIG. 1 are provided for simplicity and not intended to exclude other steps, zones, or process streams not shown.

As shown in FIG. 1, the refinery may comprise at least one distillation column. In one or more embodiments, the at least one distillation column comprises an atmospheric distillation column of an atmospheric distillation unit (ADU), and/or at least one vacuum distillation column of a vacuum distillation unit (VDU). A stream of crude oil may be introduced into the atmospheric distillation unit (ADU) and separated in the at least one atmospheric distillation column to provide several hydrocarbon fractions having specified cut points. As used herein, the term “cut point” refers to the range of temperatures over which a specified petroleum fraction boils. The lower value in a boiling point range is the initial boiling point (IBP) temperature for that specified fraction and the higher value is the end point (EP) temperature for that specified fraction. Cut points are often used to identify specific streams or fractions within and/or produced by the refinery.

In addition to a stream of crude oil, the refinery shown in FIG. 1 can also process a stream of r-pyoil introduced into the ADU. In one embodiment or in combination with any embodiments mentioned herein, the r-pyoil may originate from a pyrolysis facility as discussed previously. The ratio of the mass flow rate of r-pyoil introduced into the ADU to the mass flow rate of petroleum oil introduced into the ADU can be at least 1:1000, at least 1:750, at least 1:500, at least 1:250, at least 1:100, at least 1:50, at least 1:25, or at least 1:10 and/or not more than 1:1, not more than 1:2, not more than 1:5, or not more than 1:10. In one or more alternate embodiments, the amount of r-pyoil introduced into the ADU can be at least 0.1, at least 0.25, at least 0.75, at least 1, at least 5, at least 10, at least 15, at least 20 weight percent and/or not more than 75, not more than 65, not more than 60, not more than 50, or not more than 45 weight percent of the total feed to the at least one distillation column.

The ADU separates feed stock (e.g., crude oil) into multiple hydrocarbon streams, or fractions. These fractions include, but are not limited to, light gas, naphtha (light and heavy), gas oil (called atmospheric gas oil, or AGO), and residue or resid. When the ADU processes at least one recycled content feedstock, such as r-pyoil, each of the products formed by the ADU may include recycled content. Thus, the ADU may provide recycled content light gas (r-light gas), recycled content naphtha (r-naphtha), recycled content atmospheric gas oil (r-AGO), and recycled content atmospheric resid (r-atmospheric resid). The mass flow rate of each stream, as well as its mass or volume in proportion to other streams depends on the operation of the ADU as well as the properties of the feedstocks being processed. As mentioned previously, other hydrocarbon streams can be produced from the ADU, but are not shown here for simplicity.

The ADU comprises at least one distillation column operated at or near atmospheric pressure. Additionally, the ADU may include other equipment such as desalters, side strippers, and reflux drums/accumulators, as well as various pumps, heat exchangers, and other auxiliary equipment needed to operate the unit.

As also shown in FIG. 1, the stream of recycled content heavy naphtha (heavy r-naphtha) may be withdrawn from the ADU and can be sent to one or more downstream locations for additional processing, storage, and/or use. This stream may also be processed to remove components such as sulfur-containing compounds, chlorine-containing compounds, and/or nitrogen before further processing and/or use.

The heaviest stream withdrawn from the ADU (i.e., the ADU bottoms) is a stream of recycled content atmospheric resid (r-atmospheric resid). In some cases, the r-atmospheric resid may be introduced into the VDU. In the VDU, further separation of various hydrocarbon fractions can be performed in a vacuum distillation column operated at pressures below atmospheric pressure. For example, in one embodiment or in combination with any embodiments mentioned herein, the overhead pressure of the vacuum distillation column can be less than 100, less than 75, less than 50, less than 40, or less than 10 mm Hg. Distilling the r-atmospheric resid at low pressure permits further recovery of lighter hydrocarbon components without cracking. The VDU provides various product streams, and when it processes a recycled content feedstock, provides recycled content products. Examples of such products include, but are not limited to, recycled content light vacuum gas oil (r-LVGO), recycled content heavy vacuum gas oil (r-HVGO), and recycled content vacuum resid (r-vacuum resid).

In one embodiment or in combination with any embodiments mentioned herein, at least a portion of one or more of the heavier hydrocarbon fractions (e.g., AGO and heavier fractions) from the ADU and/or VDU can be sent to a gas oil cracker. Such heavier hydrocarbon fractions can have a median boiling point (T₅₀) greater than 375, greater than 400, greater than 500, greater than 600, greater than 700, greater than 800, or greater than 900 °F. One or more of these heavy hydrocarbon fractions can comprise at least 85, at least 90, at least 95, at least 97, or at least 99 weight percent of C10 (C15 , C20, or C25) and heavier components. Examples of these streams as shown in FIG. 1 can include, but are not limited to, r-AGO, r-atmospheric resid, r-LVGO, and r-HVGO.

The gas oil cracker can be any processing unit or zone that reduces the molecular weight of a heavy hydrocarbon feedstock to provide one or more lighter hydrocarbon products. The gas oil cracker may employ a thermal and/or catalytic cracking operation and can include other equipment such as compressors, distillation columns, heat exchangers, and other equipment necessary to provide the cracked product streams. Examples of gas oil crackers that may be used include a fluidized catalytic cracker (FCC), a steam cracker, and a hydrocracker (HDC).

In one embodiment or in combination with any embodiments mentioned herein, at least one of the heavy hydrocarbon streams introduced into and/or at least one of the cracked hydrocarbon streams removed from one or more of the gas oil crackers (e.g., hydrocracker, steam cracker, and/or FCC) may be treated with hydrogen within a hydrotreating unit (HDT) to remove all or a portion of one or more components such as sulfur-containing compounds (e.g., hydrogen sulfide, mercaptans, etc.), nitrogen-containing compounds, metals (e.g., vanadium, mercury, etc.), and/or chlorine-containing compounds and/or to saturate at least a portion of the olefinic and/or aromatic compounds in the stream. Specific locations of the hydrotreating steps can vary and may depend on the specific refinery configuration, as well as the final product specifications for sulfur, nitrogen, metal, and aromatics/olefins.

Additionally, or alternatively, one or more processing steps may be present in the refinery to remove chlorine-containing compounds. The total content of chlorine-containing compounds in the r-pyoil (or combined r-pyoil and crude oil) stream can be at least 20, at least 50, at least 75, at least 100 ppm by weight and/or not more than 500, not more than 350, not more than 200, or not more than 100 ppm by weight.

In one embodiment or in combination with any embodiments mentioned herein, at least a portion of a r-AGO stream may be combined with at least a portion of an r-LVGO stream prior to being fed to a gas oil cracker. Alternatively, these streams may be separately fed to the gas oil cracker. In one or more embodiments, the r-AGO and r-LVGO streams, merged or unmerged, are first sent to a hydrotreating unit (HDT), or particularly a fluidized catalytic cracker hydrotreating unit (FCC HDT) before being delivered to an FCC.

Alternatively, or in addition, at least a portion of the cracking of the r-AGO, r-LVGO, and/or r-HVGO streams, or portions thereof, can be performed in the presence of hydrogen (e.g., in a hydrocracker) so that removal of components such as nitrogen-, chlorine, and sulfur-containing components (and optionally metals), may be performed at the same time as the cracking reaction. When cracking and hydrogenation occur simultaneously, the saturation of olefinic hydrocarbons may also take place such that the amount of olefins in the hydrocracker product stream is not more than 20, not more than 15, not more than 10, or not more than 5 weight percent. However, aromatics may remain such that the amount of aromatics in the stream withdrawn from the hydrocracker may be at least 1, at least 5, at least 10, at least 20, or at least 25 and/or not more than 50, not more than 40, or not more than 35 weight percent. If the cracking operation is performed within a hydrocracker or the streams to be cracked have been hydrotreated prior to cracking, further downstream hydrotreating is optional.

In one embodiment or in combination with any embodiments mentioned herein, the streams exiting the gas oil cracker comprise a recycled content naphtha (r-naphtha) stream. The r-naphtha stream comprises at least 20, at least 25, at least 30, at least 35, at least 40, at least 45, or at least 50 weight percent and/or not more than 85, not more than 80, not more than 75, not more than 70, not more than 65, or not more than 60 weight percent of recycled content benzene, recycled content toluene, and recycled content xylenes (r-BTX). In one embodiment or in combination with any embodiments mentioned herein, the r-naphtha can also include at least 5, at least 10, or at least 15 weight percent and/or not more than 45, not more than 35, not more than 30, or not more than 25 weight percent of recycled content C9 to C12 aromatics and/or recycled content C6 and heavier cyclic hydrocarbons (r-C6+ cyclic hydrocarbons).

In one embodiment or in combination with any embodiments mentioned herein, the r-BTX in the r-naphtha can include at least 25, at least 30, at least 35, at least 40, or at least 45 weight percent and/or not more than 75, not more than 70, not more than 65, not more than 60, not more than 55, or not more than 50 weight percent of benzene, and/or at least 15, at least 20, at least 25, or at least 30 weight percent and/or not more than 65, not more than 60, not more than 55, not more than 50, not more than 45, not more than 40, or not more than 35 weight percent of toluene. Additionally, or in the alternative, the r-BTX in the r-reformate can include at least 5, at least 10, at least 15, or at least 20 weight percent and/or not more than 50, not more than 45, not more than 35, not more than 30, or not more than 25 weight percent of mixed xylenes, including ortho-xylene (oX), meta-xylene (mX), and para-xylene (pX). At least a portion of the benzene, toluene, and/or xylenes in the r-BTX can comprise recycled content benzene, recycled content toluene, and/or recycled content xylenes, while, in other cases, at least a portion of the benzene, toluene, and/or xylenes may include non-recycled content.

At least a portion of the recycled content naphtha comprising a hydrotreated heavy naphtha cut from the ADU, or from one or more of the cracker facilities (e.g., the FCC, steam cracker, or hydrocracker), or any combination thereof, is then fed to a reformer where the naphtha is reformed into a reformate stream. In one or more embodiments or in combination with any embodiment mentioned herein, at least a portion of the naphtha stream that undergoes reforming comprises less than 500 ppm, less than 250 ppm, less than 100 ppm, less than 75 ppm, less than 50 ppm, less than 25 ppm, or less than 10 ppm sulfur. In one or more embodiments, at least a portion of the naphtha stream that undergoes reforming comprises less than 300 ppm, less than 150 ppm, less than 100 ppm, less than 50 ppm, less than 25 ppm, less than 10 ppm, or less than 5 ppm chlorine. In one or more embodiments, at least a portion of the naphtha stream that undergoes reforming comprises less than 500 ppm, less than 250 ppm, less than 100 ppm, less than 75 ppm, less than 50 ppm, less than 30 ppm, or less than 20 ppm water. In one or more embodiments, at least a portion of the naphtha stream that undergoes reforming comprises less than 500 ppb, less than 250 ppb, less than 100 ppb, less than 50 ppb, less than 25 ppb, less than 10 ppb, less than 5 ppb, or less than 2 ppb arsenic.

Turning again to FIG. 1, at least a portion of any r-naphtha withdrawn from the one of the crackers can be combined with any other portion of r-naphtha streams from any other gas oil crackers and introduced into an aromatics complex, wherein the stream can be processed to provide a recycled content paraxylene (r-paraxylene) stream. The r-paraxylene stream, which comprises recycled content paraxylene (r-pX), can also include non-recycled content components, including non-recycled content paraxylene (pX). The r-paraxylene stream can include at least 55, at least 60, at least 65, at least 70, at least 75, at least 80, at least 85, at least 90, at least 95, at least 97, or at least 99 percent of r-pX, based on the total amount of r-pX and pX in the stream. The total amount of paraxylene in the r-paraxylene stream (including both pX and r-pX) can be at least 85, at least 90, at least 92, at least 95, at least 97, at least 99,or at least 99.5 weight percent. In some cases, all of the paraxylene in the r-paraxylene stream can be r-pX.

Referring now to FIG. 2, an alternative process for producing r-pX is illustrated. The process of FIG. 2 is similar to that depicted in FIG. 1 in that a recycled content pyoil feed and a crude oil feed are processed within at least one distillation column within the refinery, and in particular, into one or more distillation columns of an ADU and/or VDU. Within these distillation units, recycled content AGO, LVGO, and/or HVGO can be produced along with a recycled content heavy naphtha cut and other lighter hydrocarbon cuts. In one or more embodiments, the recycled content heavy hydrocarbon streams can be individually fed, or combined and fed, to a cracking facility, such as a steam cracking facility, in which the average molecular weight of the heavy hydrocarbon streams are reduced to produce a recycled content pyrolysis gasoline stream.

In one embodiment or in combination with any embodiments mentioned herein, the r-pyrolysis gasoline stream comprises at least 20, at least 25, at least 30, at least 35, at least 40, at least 45, or at least 50 weight percent and/or not more than 85, not more than 80, not more than 75, not more than 70, not more than 65, or not more than 60 weight percent of recycled content benzene, recycled content toluene, and recycled content xylenes (r-BTX). In one embodiment or in combination with any embodiments mentioned herein, the r-pyrolysis gasoline can also include at least 5, at least 10, or at least 15 weight percent and/or not more than 45, not more than 35, not more than 30, or not more than 25 weight percent of recycled content C9 to C12 aromatics and/or recycled content C6 and heavier cyclic hydrocarbons (r-C6+ cyclic hydrocarbons).

In one embodiment or in combination with any embodiments mentioned herein, the r-BTX in the r-pyrolysis gasoline can include at least 25, at least 30, at least 35, at least 40, or at least 45 weight percent and/or not more than 75, not more than 70, not more than 65, not more than 60, not more than 55, or not more than 50 weight percent of benzene, and/or at least 15, at least 20, at least 25, or at least 30 weight percent and/or not more than 65, not more than 60, not more than 55, not more than 50, not more than 45, not more than 40, or not more than 35 weight percent of toluene. Additionally, or in the alternative, the r-BTX in the r-pyrolysis gasoline can include at least 5, at least 10, at least 15, or at least 20 weight percent and/or not more than 50, not more than 45, not more than 35, not more than 30, or not more than 25 weight percent of mixed xylenes, including ortho-xylene (oX), meta-xylene (mX), and para-xylene (pX). At least a portion of the benzene, toluene, and/or xylenes in the r-BTX can comprise recycled content benzene, recycled content toluene, and/or recycled content xylenes, while, in other cases, at least a portion of the benzene, toluene, and/or xylenes may include non-recycled content.

At least a portion of the r-pyrolysis gasoline stream is fed to a hydrogenation unit in which unsaturated carbon-carbon bonds are reduced in the presence of hydrogen to form saturated carbon-carbon bonds. The hydrogenation unit can employ one or more hydrogenation reactors that contain a catalyst, such as nickel, palladium, rhodium, ruthenium, or platinum-containing catalysts. At least a portion of the hydrogenated r-pyrolysis gasoline stream is then fed to an aromatics complex.

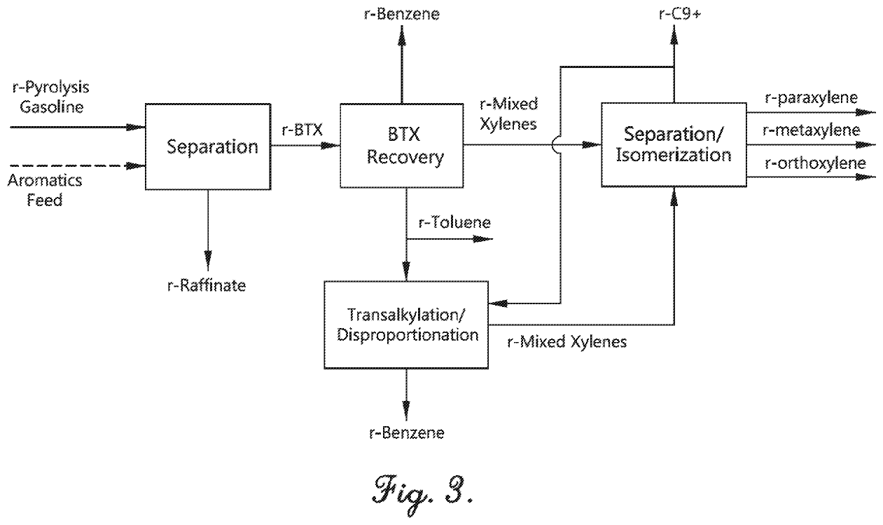

As depicted in FIG. 3, and described in further detail below, the aromatics complex may produce an r-raffinate stream from a separation section, and in particular, from an extraction unit. The r-raffinate stream is depleted in aromatics and comprises predominantly C5 to C12 components and may include not more than 20, not more than 15, not more than 10, or not more than 5 weight percent of C6 to C9 aromatics (e.g., benzene, toluene, and xylenes). The r-raffinate stream, which comprises at least a portion of the r-pyrolysis gasoline stream, may be combined with at least a portion of a heavy r-naphtha stream produced from the ADU. The r-raffinate stream may be combined with the heavy r-naphtha stream and the combined stream hydrotreated. Alternatively, the r-raffinate stream (and optionally the heavy r-naphtha stream as well) may bypass hydrotreatment and be directed to a reformer unit. Thus, at least a portion of the r-pyrolysis gasoline stream, namely that portion which comprises the r-raffinate stream, is indirectly reformed within the reformer unit.

As described with regard to FIG. 1, the reforming reforms the combined heavy r-naphtha and r-raffinate streams into an r-reformate stream comprising, among other things, r-pX. The r-reformate stream may be combined with the hydrogenated r-pyrolysis gasoline stream and then be fed to the aromatics complex, as described below.

Referring now to FIG. 3, a schematic diagram of the main steps/zones of an aromatics complex as shown in FIGS. 1 and 2 is provided. As shown in FIG. 3, at least a portion of an r-reformate stream from a reforming unit can be introduced into an initial separation step in the aromatics complex.

The initial separation step shown in FIG. 3 for removing BTX from the incoming streams may be performed using any suitable type of separation, including extraction, distillation, and extractive distillation. When the separation step includes extraction or extractive distillation, it may utilize at least one solvent selected from the group consisting of sulfolane, furfural, tetraethylene glycol, dimethylsulfoxide, N,N-dimethylformamide, and N-methyl-2-pyrrolidone. Upon separation, a recycled content raffinate (r-raffinate) stream depleted in aromatics can be withdrawn from the separation step/zone. The r-raffinate stream comprises predominantly C5 to C12 components and may include not more than 20, not more than 15, not more than 10, or not more than 5 weight percent of C6 to C9 aromatics (e.g., benzene, toluene, and xylenes).

Additionally, as shown in FIG. 3, a stream concentrated in recycled content benzene, toluene, and xylenes (r-BTX) can also be withdrawn from the initial separation step. This r-BTX stream comprises predominantly BTX and may include at least 60, at least 70, at least 80, at least 85, at least 90, or at least 95 BTX, including both recycled content BTX (r-BTX) and non-recycled content BTX, as applicable. The r-BTX stream can be introduced into a downstream separation train, also referred to as a BTX recovery zone, which utilizes one or more separation steps to provide streams concentrated in recycled content benzene (r-benzene), recycled content mixed xylenes (r-mixed xylenes), and recycled content toluene (r-toluene). Such separations can be performed according to any suitable method, including, for example, with one or more distillation columns or other separation equipment or steps.

As shown in FIG. 3, the r-benzene formed in BTX recovery step can be removed as a product stream from aromatics complex, while the r-mixed xylenes can be introduced into a second separation train for separating out recycled content ortho-xylene (r-oX), recycled content meta-xylene (r-mX), and/or recycled content paraxylene (r-pX) from the other components in the stream. Additionally, at least a portion of the ox (or r-oX) and/or mX (or r-mX) can be subjected to isomerization to provide additional pX (or r-pX). After the isomerization, additional separation steps may be performed to provide individual streams of oX (or r-oX), mX (or r-mX), and pX (or r-pX). This second separation step can utilize one or more of distillation, extraction, crystallization, and adsorption to provide recycle content aromatics streams. For example, as shown in FIG. 3, the separation step can provide at least one of a recycled content paraxylene (r-paraxylene) stream, a recycled content metaxylene (r-metaxylene) stream, and a recycled content orthoxylene (r-orthoxylene) stream. Each of these streams may include both recycled and non-recycled content and can individually include at least 75, at least 80, at least 85, at least 90, at least 95, or at least 97 weight percent of paraxylene (r-pX and pX), metaxylene (r-mX and mX), or orthoxylene (r-oX and oX), respectively.

Additionally, as shown in FIG. 3, a stream of recycled content C9 and heavier components (r-C9+ components) may also be withdrawn from the second separation step and all or a portion may be introduced into a transalkylation/disproportionation step along with a stream of r-toluene withdrawn from the BTX recovery step/zone. In the transalkylation/disproportionation step/zone, at least a portion of the toluene (or r-toluene) can be reacted in the presence of a regenerable fixed bed silica-alumina catalyst to provide mixed xylenes (or r-mixed xylenes) and benzene (or r-benzene). Alternatively, or in addition, at least a portion of the r-toluene can be reacted with methanol (and, optionally, r-methanol) to provide recycled content paraxylene (r-paraxylene), which may be further processed as described herein. In some cases, this reaction may be performed within the aromatics complex over an acidic catalyst, preferably on a shape-selective molecular sieve catalyst such as ZSM-5, and the resulting r-paraxylene may be combined with other paraxylene (or r-paraxylene) recovered in the aromatics complex, as shown in FIG. 3. As also shown in FIG. 3, the benzene (or r-benzene) can be recovered as a product, while the r-mixed xylenes can be introduced into the second separation step/zone for further separation into a r-paraxylene stream, an r-orthoxylene stream, and a r-metaxylene stream.

At least a portion of the r-paraxylene stream withdrawn from the aromatics complex can be sent to a TPA production facility. In the TPA production facility, at least a portion of the pX (and/or r-pX) in the r-paraxylene stream can be oxidized in the presence of a solvent (e.g., acetic acid) and a catalyst to form recycled content crude terephthalic acid (r-CTA).

Thereafter, depending on the specific TPA production process utilized within the production facility, the r-CTA can either be oxidized again in a secondary or post-oxidation step or it can be hydrogenated in a treatment step to form recycled content purified terephthalic acid (r-PTA). All or a portion of the solvent may be removed from the r-CTA and swapped out for new solvent, which may be the same as or different than the original solvent. The resulting r-PTA slurry can be processed by, for example, drying, crystallization, and filtration to provide the final r-TPA product.

In one embodiment or in combination with any embodiments mentioned herein, at least a portion of the r-TPA product can be introduced into a PET production facility and reacted with at least one diol (such as, for example, ethylene glycol) to form recycled content polyethylene terephthalate (r-PET). In one embodiment or in combination with any embodiments mentioned herein, the r-TPA and ethylene glycol (or, recycled content ethylene glycol, r-EG) can be polymerized in the presence of one or more comonomers, such as isophthalic acid or neopentyl glycol or cyclohexanedimethanol, to form a recycled content PET copolymer (r-co-PET).

### Definitions

It should be understood that the following is not intended to be an exclusive list of defined terms. Other definitions may be provided in the foregoing description, such as, for example, when accompanying the use of a defined term in context.

As used here, the term “light gas” refers to a hydrocarbon-containing stream comprising at least 50 weight percent of C4 and lighter hydrocarbon components. Light hydrocarbon gas may include other components such as nitrogen, carbon dioxide, carbon monoxide, and hydrogen, but these are typically present in amount of less than 20, less than 15, less than 10, or less than 5 weight percent, based on the total weight of the stream.

As used herein, the terms “median boiling point” or “T50” refers to the median boiling point of a process stream (i.e., the temperature value where 50 weight percent of the stream composition boils above the temperature value and 50 weight percent of the stream composition boils below the temperature value).

As used herein, the term “boiling point range” or “cut point” refers to the range of temperatures over which a specified petroleum fraction boils. The lower value in a boiling point range is the initial boiling point (IBP) temperature for that specified fraction and the higher value is the end point (EP) temperature for that specified fraction.

As used herein, the term “naphtha” refers to a physical mixture of hydrocarbon components separated in at least one distillation column of a refining facility that has a boiling point range between 90 to 380 °F.

As used herein, the term “light naphtha” refers to a specific portion of a naphtha cut in a refinery having a boiling point range between 90 and 190 °F.

As used herein, the term “heavy naphtha” refers to a specific portion of a naphtha cut in a refinery having a boiling point range between 190 and 380 °F.

As used herein, the term “gas oil” refers to a physical mixture of hydrocarbon components separated in at least one distillation column of a refining facility that has a boiling point range greater than 520 to 1050 °F.

As used herein, the term “atmospheric gas oil” refers to a gas oil produced by the atmospheric distillation unit.

As used herein, the term “light gas oil” or “LGO” refers to a specific portion of gas oil cut in a refinery having a boiling point range between greater than 520 and 610 °F.

As used herein, “light vacuum gas oil” or “LVGO” refers to a light gas oil produced by the vacuum distillation unit.

As used herein, the term “heavy gas oil” or “HGO” refers to a specific portion of a gas oil cut in a refinery having a boiling point range between greater than 610 and 800 °F.

As used herein, “heavy vacuum gas oil” or “HVGO” refers to a heavy gas oil produced by the vacuum distillation unit.

As used herein, the term “vacuum gas oil” or “VGO” refers to a specific portion of a gas oil cut in a refinery having a boiling point range between greater than 800 and 1050 °F. Vacuum gas oil is separated from the initial crude oil using a vacuum distillation column operated at a pressure below atmospheric pressure.

As used herein, the term “residue” or “resid” refers to the heaviest cut from a distillation column in a refinery and having a boiling point range between greater than 1050 °F.

As used herein, the term “vacuum resid” refers to a resid product from the vacuum distillation column.

As used herein, the term “atmospheric resid” refers to a resid product from the atmospheric distillation column.

As used herein, the term “gas oil cracker” refers to a cracking unit for processing a feed stream comprising predominantly gas oil and heavier components. Although a gas oil cracker can process lighter components, such as distillate and naphtha, at least 50 weight percent of the total feed to a gas oil cracker includes gas oil and heavier components. Gas oil crackers may be operated at temperatures of at least 350 °F, at least 400 °F, at least 450 °F, at least 500 °F, at least 550 °F, or at least 600 °F and/or not more than 1200 °F, not more than 1150 °C, not more than 1100 °F, not more than 1050 °F, not more than 1000 °F, not more than 900 °F, or not more than 800 °F. Gas oil crackers may be operated at or near atmospheric pressure (e.g., at a pressure of less than 5 psig, less than 2 psig, or 1 psig) or may be operated at elevated pressure (e.g., at a pressure of at least 5 psig, at least 10 psig, at least 25 psig, at least 50psig, at least 100 psig, at least 250 psig, at least 500 psig, or at least 750 psig.) Additionally, the cracking in gas oil crackers may be carried with or without a catalyst, and it may or may not be conducted in the presence of hydrogen and/or steam.

As used herein, the term “fluidized catalytic cracker” or “FCC” refers to a set of equipment, including a reactor, a regenerator, a main fractionator, as well as ancillary equipment such as pipes, valves, compressors, and pumps, for reducing the molecular weight of a heavy hydrocarbon stream via catalytic cracking in a fluidized catalyst bed.

As used herein, the terms “reformer” or “catalytic reformer” refer to a process or facility in which a feedstock comprising predominantly C6-C10 alkanes is converted to a reformate comprising branched hydrocarbons and/or cyclic hydrocarbons in the presence of a catalyst.

As used herein, the term “reformate” refers to a liquid product stream produced by a catalytic reformer process.

As used herein, the term “hydroprocessing” refers to chemical processing of a hydrocarbon stream with or in the presence of hydrogen. Hydroprocessing is typically a catalytic process and includes hydrocracking and hydrotreating.

As used herein, the term “hydrocracking” refers a type of hydroprocessing where the hydrocarbon molecules are cracked (i.e., undergo a reduction in molecular weight).

As used herein, the term “hydrotreating” refers to a type of hydroprocessing that does not crack the hydrocarbon molecules, but instead removes oxygen, sulfur, and other heteroatoms by hydrogenolysis or to saturate unsaturated bonds by hydrogenation. It may or may not be carried out in the presence of a catalyst.

As used herein, the term “distillation” refers to separation of a mixture of components by boiling point difference.

As used herein, the term “atmospheric distillation” refers to distillation performed at a pressure at or near atmospheric, usually to separate crude oil and/or other streams into specified fractions for further processing.

As used herein, the term “vacuum distillation” refers to distillation performed at a pressure below atmospheric and, usually, at a pressure of less than 100 mm Hg at the top of the column.

As used herein, the term “aromatics complex” refers to a process or facility in which a mixed hydrocarbon feedstock, such as a reformate, is converted into one or more benzene, toluene, and/or xylene (BTX) product streams, such as a para-xylene product stream. The aromatics complex may comprise one or more processing steps, in which one or more components of the reformate are subjected to at least one of a separation step, an alkylating step, a transalkylating step, a toluene disproportionation step, and/or an isomerization step. The separation step can comprise one or more of an extraction step, a distillation step, a crystallization step, and/or an adsorption step.

As used herein, the term “raffinate” refers to the aromatics-depleted stream removed from the initial separation step in the aromatics complex. Although most commonly used to refer to a stream withdrawn from an extraction step, the term “raffinate” as used with respect to the aromatics complex can also refer to a stream withdrawn from another type of separation, including, but not limited to, distillation or extractive distillation.

As used herein, the terms “recycled content pyrolysis oil” or “r-pyoil” refers to a composition that is directly or indirectly derived from pyrolysis of a waste plastic that is liquid at 25 °C and 1 atm, absolute.

As used herein, the terms “pyrolysis gas” and “pygas” refer to a composition obtained from pyrolysis that is gaseous at 25 °C and 1 atm, absolute.

As used herein, the term “pyrolysis” refers to thermal decomposition of one or more organic materials at elevated temperatures in an inert (i.e., substantially oxygen free) atmosphere.

As used herein, the term “pyrolysis vapor” refers to the overhead or vapor-phase stream withdrawn from the separator in a pyrolysis facility used to remove r-pyrolysis residue from the r-pyrolysis effluent.

As used herein, the term “pyrolysis effluent” refers to the outlet stream withdrawn from the pyrolysis reactor in a pyrolysis facility.

As used herein, the term “r-pyrolysis residue” refers to a composition obtained from waste plastic pyrolysis that comprises predominantly pyrolysis char and pyrolysis heavy waxes.

As used herein, the term “pyrolysis char” refers to a carbon-containing composition obtained from pyrolysis that is solid at 200 °C and 1 atm, absolute.

As used herein, the term “pyrolysis gasoline” refers to a hydrocarbon stream of predominantly C5 and heavier components removed from a quench section of a steam cracking facility. Typically, pyrolysis gasoline includes at least 10 weight percent of C6 to C9 aromatics.

As used herein, the term “pyrolysis heavy waxes” refers to C20+ hydrocarbons obtained from pyrolysis that are not pyrolysis char, pyrolysis gas, or pyrolysis oil.

As used herein, the term “lighter” refers to a hydrocarbon component or fraction having a lower boiling point than another hydrocarbon component or fraction.

As used herein, the term “heavier” refers to a hydrocarbon component or fraction having a higher boiling point than another hydrocarbon component or fraction.

As used herein, the term “upstream” refers to an item of facility that is positioned prior to another item or facility in a given process flow and may include intervening items and/or facilities.

As used herein, the term “downstream” refers to an item or facility that is positioned after another item or facility in a given process flow and may include intervening items and/or facilities.

As used herein, the term “alkane” refers to a saturated hydrocarbon including no carbon-carbon double bonds.

As used herein, the term “olefin” refers to an at least partially unsaturated hydrocarbon including at least one carbon-carbon double bond.

As used herein, the terms “Cx” or “Cx hydrocarbon” or “Cx component” refers to a hydrocarbon compound including “x” total carbons per molecule, and encompasses all olefins, paraffins, aromatics, heterocyclic, and isomers having that number of carbon atoms. For example, each of normal, iso, and tert-butane and butene and butadiene molecules would fall under the general description “C4” or “C4 components.”

As used herein, the terms “r-para-xylene” or “r-pX” refer to being or comprising a para-xylene product that is directly and/or indirectly derived from waste plastic.

As used herein, the term “cracking” refers to breaking down complex organic molecules into simpler molecules by the breaking of carbon-carbon bonds.

As used herein, the term “steam cracking” refers to thermal cracking of hydrocarbons in the presence of steam, usually performed in a furnace of the steam cracking facility.

As used herein, the term “co-located” refers to the characteristic of at least two objects being situated on a common physical site, and/or within five miles of each other, measured as a straight-line distance between two designated points.

As used herein, the term “commercial scale facility” refers to a facility having an average annual feed rate of at least 500 pounds per hour, averaged over one year.

As used herein, the terms “crude” and “crude oil” refer to a mixture of hydrocarbons that exists in liquid phase and is derived from natural underground reservoirs.

As used herein, the terms “recycle content” and “r-content” refer to being or comprising a composition that is directly and/or indirectly derived from waste plastic.

As used herein, the term “predominantly” means more than 50 percent by weight. For example, a predominantly propane stream, composition, feedstock, or product is a stream, composition, feedstock, or product that contains more than 50 weight percent propane.

As used herein, the term “waste material” refers to used, scrap, and/or discarded material.

As used herein, the terms “waste plastic” and “plastic waste” refer to used, scrap, and/or discarded plastic materials, including post-industrial or pre-consumer waste plastic and post-consumer waste plastic.

As used herein, the terms “mixed plastic waste” and “MPW” refer to a mixture of at least two types of waste plastics including, but not limited to the following plastic types: polyethylene terephthalate (PET), one or more polyolefins (PO), and polyvinylchloride (PVC).

As used herein, the term “fluid communication” refers to the direct or indirect fluid connection between two or more processing, storage, or transportation facilities or zones.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the terms “including,” “include,” and “included” have the same open-ended meaning as “comprising,” “comprises,” and “comprise” provided above.

As used herein, the term “predominantly” means more than 50 percent by weight. For example, a predominantly propane stream, composition, feedstock, or product is a stream, composition, feedstock, or product that contains more than 50 weight percent propane.

As used herein, the term “chemical pathway” refers to the chemical processing step or steps (e.g., chemical reactions, physical separations, etc.) between an input material and a product, where the input material is used to make the product.

As used herein, the term “mass balance” refers to a method of tracing recycled content based on the mass of the recycled content in a product.

As used herein, the terms “physical recycled content” and “direct recycled content” both refer to matter physically present in a product and that is physically traceable back to a waste material.

As used herein, the term “recycled content” refers to being or comprising a composition that is directly and/or indirectly derived from recycled waste material. Recycled content is used generically to refer to both physical recycled content and credit-based recycled content. Recycled content is also used as an adjective to describe a product having physical recycled content and/or credit-based recycled content.

As used herein, the term “recycled content credit” refers to a non-physical measure of physical recycled content obtained from a mass of waste plastic that can be directly or indirectly (i.e., via a digital inventory) attributed to a product second material.

As used herein, the term “hydroprocessing unit” refers to a set of equipment, including reaction vessels, a drier, and a main fractionator, as well as ancillary equipment such as pipes, valves, compressors, and pumps, for chemically processing a hydrocarbon stream in the presence of hydrogen. Specific examples of hydroprocessing units include a hydrocracker (or hydrocracking unit) configured to carry out a hydrocracking process and a hydrotreater (or hydrotreating unit) configured to carry out a hydrotreating process.

As used herein, the term “coker” or “coking unit” refers to a set of equipment, including reaction vessels, a drier, and a main fractionator, as well as ancillary equipment such as pipes, valves, compressors, and pumps, for reducing the molecular weight of a heavy hydrocarbon stream via thermal cracking or coking.

As used herein, the terms “steam cracking facility” or “steam cracker” refer to all of the equipment needed to carry out the processing steps for thermally cracking a hydrocarbon feed stream in the presence of steam to form one or more cracked hydrocarbon products. Examples include, but are not limited to, olefins such as ethylene and propylene. The facility may include, for example, a steam cracking furnace, cooling equipment, compression equipment, separation equipment, as well as the pipes, valves, tanks, pumps, etc. needed to carry out the processing steps.

As used herein, the terms “refinery,” “refining facility,” and “petroleum refinery,” refer to all of the equipment needed to carry out the processing steps for separating and converting petroleum crude oil into multiple hydrocarbon fractions, one or more of which can be used as a fuel source, lube oil, bitumen, coke, and as an intermediate for other chemical products.” The facility may include, for example, separation equipment, thermal or catalytic cracking equipment, chemical reactors, and product blending equipment, as well as the pipes, valves, tanks, pumps, etc. needed to carry out the processing steps.

As used herein, the term “pyrolysis facility,” refers to all of the equipment needed to carry out the processing steps for pyrolyzing a hydrocarbon-containing feed stream, which can include or be waste plastic. The facility may include, for example, reactors, cooling equipment, and separation equipment, as well as the pipes, valves, tanks, pumps, etc. needed to carry out the processing steps.

As used herein, the term “terephthalic acid production facility,” or “TPA production facility,” refers to all of the equipment needed to carry out the processing steps for forming terephthalic acid from paraxylene. The facility may include, for example, reactors, separators, cooling equipment, separation equipment such as filters or crystallizers, as well as the pipes, valves, tanks, pumps, etc. needed to carry out the processing steps.

As used herein, the term “polyethylene terephthalate production facility,” or “PET production facility,” refers to all of the equipment needed to carry out the processing steps for forming polyethylene terephthalate (PET) from a terephthalate, ethylene glycol, and, optionally, one or more additional monomers. The facility may include, for example, polymerization reactors, cooling equipment, and equipment to recover solidified and/or pelletized PET, as well as the pipes, valves, tanks, pumps, etc. needed to carry out the processing steps.

### Claims not Limited to Disclosed Embodiments

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A method of producing r-p-xylene comprising:
(a) distilling an r-pyoil stream and a crude oil stream in at least one distillation column to produce at least one r-heavy hydrocarbons stream having a T50 of greater than 400 °F;
(b) reducing an average molecular weight of said at least one r-heavy hydrocarbons stream to produce an r-naphtha stream and/or r-pyrolysis gasoline stream; and
(c) directly or indirectly reforming at least a portion of said r-naphtha stream and/or r-pyrolysis gasoline stream in a reformer to produce an r-reformate comprising said r-p-xylene.

2. The method of claim 1, wherein said at least one distillation column comprises an atmospheric distillation column and a vacuum distillation column, and wherein a bottoms stream from said atmospheric distillation column comprises a feed to said vacuum distillation column, wherein said atmospheric distillation column produces at least an r-atmospheric gas oil stream, and wherein said vacuum distillation column produces at least an r-light vacuum gas oil stream and an r-heavy vacuum gas oil stream.

3. The method of claim 2, wherein at least a portion of said r-atmospheric gas oil stream and at least a portion of said r-light vacuum gas oil stream comprise said r-heavy hydrocarbons stream.

4. The method of claim 2, wherein at least a portion of said r-heavy vacuum gas oil stream comprises said r-heavy hydrocarbons stream.

5. The method of claim 1, wherein at least a portion of said r-naphtha stream undergoes hydrotreating prior to being reformed.

6. The method of claim 1, wherein (b) comprises subjecting at least a portion of said r-heavy hydrocarbons stream to a cracking operation within a cracking facility, wherein said cracking operation comprises steam cracking, hydrocracking and/or fluidized catalytic cracking.

7. The method of claim 1, wherein at least a portion of said r-reformate comprising said recycled-content p-xylene and/or r-pyrolysis gasoline stream is processed within an aromatics complex to produce an r-p-xylene-containing product stream, wherein said product stream comprises at least 90 wt. % p-xylene.

8. A method of producing r-p-xylene comprising:
(a) distilling an r-pyoil stream and a crude oil stream within an atmospheric distillation column and a vacuum distillation column to produce an r-atmospheric gas oil stream, an r-light vacuum gas oil stream, and an r-heavy vacuum gas oil stream;
(b) reducing an average molecular weight of said r-heavy vacuum gas oil stream within a hydrocracker to produce a first r-naphtha stream;
(c) hydrotreating at least a portion of at least one of said r-light vacuum gas oil stream and said r-atmospheric gas oil stream to produce an r-hydrotreated stream, and reducing an average molecular weight of said r-hydrotreated stream within a fluidized catalytic cracker to produce a second r-naphtha stream;
(d) optionally, hydrotreating at least a portion of said first and/or second r-naphtha streams to produce a hydrotreated r-naphtha stream; and
(e) reforming at least a portion of said first and/or second r-naphtha streams and/or said optionally hydrotreated r-naphtha stream to produce an r-reformate comprising said r-p-xylene.

9. The method of claim 8, wherein at least a portion of said r-reformate comprising said r-p-xylene is processed within an aromatics complex to produce an r-p-xylene-containing product stream, wherein said product stream comprises at least 90 wt. % p-xylene.

10. The method of claim 8, wherein at least a portion of said r-light vacuum gas oil stream and/or at least a portion of said r-atmospheric gas oil stream are fed to said hydrocracker.

11. A method of producing an r-terephthalic acid comprising oxidizing an r-p-xylene, wherein said r-p-xylene is obtained by:
(a) distilling an r-pyoil stream and a crude oil stream in at least one distillation column to produce at least one r-heavy hydrocarbons stream having a T50 of greater than 400 °F;
(b) reducing an average molecular weight of said r-heavy hydrocarbons stream to produce an r-naphtha stream and/or an r-pyrolysis gasoline stream; and
(c) directly or indirectly reforming at least a portion of said r-naphtha stream and/or at least a portion of said r-pyrolysis gasoline stream in a reformer to produce an r-reformate comprising said r-p-xylene.

12. The method of claim 11, wherein said at least one distillation column comprises an atmospheric distillation column and a vacuum distillation column, and wherein a bottoms stream from said atmospheric distillation column comprises a feed to said vacuum distillation column, and wherein said atmospheric distillation column produces at least an r-atmospheric gas oil stream, and wherein said vacuum distillation column produces at least an r-light vacuum gas oil stream, at least a portion of said r-atmospheric gas oil stream and at least a portion of said r-light vacuum gas oil stream comprising said r-heavy hydrocarbons stream.

13. The method of claim 12, wherein said vacuum distillation column further produces an r-heavy vacuum gas oil stream, said r-heavy vacuum gas oil stream comprising said r-heavy hydrocarbons stream.

14. The method of claim 11, wherein at least a portion of said r-naphtha stream does not undergo hydrotreating prior to being reformed.

15. (canceled)

16. The method of claim 11, wherein (b) comprises subjecting at least a portion of said r-heavy hydrocarbons stream to a cracking operation within a cracking facility, wherein said cracking operation comprises steam cracking, hydrocracking, and/or fluidized catalytic cracking.

17. The method of claim 11, wherein at least a portion of said r-reformate and/or at least a portion of said r-pyrolysis gasoline comprising said r-p-xylene are processed within an aromatics complex to produce an r-p-xylene-containing product stream, wherein said product stream comprises at least 90 wt. % p-xylene.

18. A method of producing r-p-xylene comprising:
(a) distilling an r-pyoil stream and a crude oil stream in at least one distillation column to produce an r-heavy naphtha stream;
(b) optionally, hydrotreating said r-heavy naphtha stream to produce a hydrotreated r-heavy naphtha stream; and
(c) reforming at least a portion of said r-heavy naphtha stream or hydrotreated r-heavy naphtha stream in a reformer to produce an r-reformate comprising said r-p-xylene.

19. The method of claim 18, wherein said at least one distillation column comprises an atmospheric distillation column and a vacuum distillation column, and wherein a bottoms stream from said atmospheric distillation column comprises a feed to said vacuum distillation column, wherein said atmospheric distillation column produces at least an r-atmospheric gas oil stream, and wherein said vacuum distillation column produces at least an r-light vacuum gas oil stream and an r-heavy vacuum gas oil stream, and wherein at least a portion of said r-atmospheric gas oil stream, at least a portion of said r-light vacuum gas oil stream, and/or at least a portion of said r-heavy vacuum gas oil stream undergo a reduction in average molecular weight to produce one or more cracked r-naphtha and/or r-pyrolysis gasoline streams, wherein said reduction in average molecular weight comprises a cracking operation, and wherein said cracking operation comprises steam cracking, hydrocracking and/or fluidized catalytic cracking.

20. The method of claim 19, wherein at least a portion of said one or more cracked r-naphtha and/or r-pyrolysis gasoline streams are combined with said r-heavy naphtha stream or said hydrotreated r-heavy naphtha stream prior to being reformed in said reformer.
